(19) United States
(12) Patent Application Publication
(10) Pub. No.: US 2008/0255565 A1
(43) Pub. Date: Oct. 16, 2008

(54) Broach handle for minimally invasive hip replacement surgery

(76) Inventor: Henry H. Fletcher, Cameron Park, CA (US)

Correspondence Address: Mark J. Pandiscio;Pandiscio & Pandiscio, P. C., 470 Totten Pond Road, Waltham, MA, 02451-1914, US

(21) Appl. No.: 11/986,277

(22) Filed: Nov. 20, 2007

Related U.S. Application Data
(60) Provisional application No. 60/860,062, filed on Nov. 20, 2006.

(51) Int. Cl. A61B 17/00

(52) U.S. Cl. 606/80

(57) ABSTRACT

A broach handle for attachment to a broach comprising a distal working end and a proximal connector end, the proximal connector end comprising a bore extending distally into the broach, and a finger comprising a recess and extending proximally away from the broach, the broach handle comprising: a body having a distal end and a proximal end, the distal end being longitudinally and laterally offset from the proximal end and comprising a finger for seating in the bore of the broach and a bore for receiving the finger of the broach; a linkage connected to the body and comprising a finger for selective disposition in the recess of the finger of the broach; and a lever connected to the body and arranged to manipulate the proximal end of the linkage so that manipulation of the proximal end of the lever can lock and unlock a broach to the handle.

### REFERENCE TO PENDING PRIOR PATENT APPLICATION

This patent application claims benefit of pending prior U.S. Provisional Patent Application Ser. No. 60/860,062, filed Nov. 20, 2006 by Henry H. Fletcher for UNIVERSAL BROACH HANDLE (Attorney's Docket No. ORTHO-1 PROV), which patent application is hereby incorporated herein by reference.

### FIELD OF THE INVENTION

This invention relates to the preparation of a bone for an implant in general, and more particularly to the preparation of the intramedullary canal of a femur for a minimally invasive total hip replacement.

### BACKGROUND OF THE INVENTION

Joint replacement surgery seeks to replace portions of a joint with prosthetic components so as to provide long-lasting function and pain-free mobility.

More particularly, in the case of a total hip replacement, the head of the femur is removed and replaced with a prosthetic femoral component, and the socket of the acetabulum is replaced by a prosthetic acetabular component, whereby to provide a prosthetic total hip.

There are several steps to performing a total hip replacement. First, the femur and the acetabulum must be prepared, then the femur and the acetabulum must be fitted for their respective prostheses, and then the prostheses must be fixed to the surrounding bone.

In order to prepare the bone for the femoral prosthesis, the head of the femur is distracted from its socket in the acetabulum, thereby exposing the femoral head and the acetabular cup. Next, a femoral neck osteotomy is performed, wherein the neck and head of the femur are removed from the remainder of the femur. Then the proximal end of the intramedullary canal is prepared to receive the stem of the prosthesis. More particularly, a rasp, reamer, broach, etc. is used to hollow, clean and enlarge the intramedullary canal of the femur so as to create a cavity that matches the shape of the stem of the prosthesis which is to be inserted into the intramedullary canal.

In cases where a broach is used to hollow, clean and enlarge the intramedullary canal, a series of successively larger broaches are used to enlarge the intramedullary canal. The broach, which generally comprises a tapered body, is removably attached to a broach handle. During use, the surgeon strikes a strike plate on the proximal end of the broach handle with a hammer so as to drive the broach into the intramedullary canal.

Correspondingly, the acetabulum is prepared to receive the acetabular prosthesis.

After the femur and the acetabulum have been prepared, the acetabular prosthesis is positioned, an appropriate femoral prosthesis is selected, the stem of the femoral prosthesis is inserted into the prepared intramedullary canal, the femoral prosthesis is fixed to the bone using cement, and then the joint is reduced so that the ball of the femoral prosthesis is seated in the acetabular prosthesis.

In order to ensure a proper fit of the femoral prosthesis with the femur, it is essential that the cavity within the femur be properly oriented and closely replicate the dimensions of the femoral prosthesis. An improper fit of the femoral prosthesis within the cavity may lead to micro-rotation of the stem relative to the femur and unstable fixation, which may cause the bone cavity to weaken and deform over time. This can present serious problems for the patient, including joint instability and pain.

Accordingly, in addition to using a broach to form a recess in the intramedullary canal, it has become a common practice to use the broach as a trial implant in order to determine the particular size and orientation of the stem component. Once the size of the trial broach is determined, the trial broach is left in the intramedullary canal and a femoral prosthesis head is placed on a post extending from the trial broach in order to check the range of motion, muscle tension, and leg length. After the size and orientation is determined, the femoral prosthesis head is removed and a handle is re-attached to the post of the trial broach. The trial broach is then removed from the bone socket and replaced with the permanent femoral prosthesis.

In order to efficiently use the broach for preparing the femoral cavity and as a trial implant, it is important that the broach handle be releasable from the broach. Traditional broach handles are difficult to detach from the broach during surgery without impinging soft tissue because the mechanisms to detach the broach handle from the broach are typically located close to the broach.

To this end, an improved handle is needed for attachment to the broach in order to enable the surgeon to effectively and accurately enlarge the intramedullary canal and properly size the hip prosthesis while minimizing tissue impingement and damage.

More particularly, a broach handle is needed which may be efficiently and deliberately detached from the broach in order to (i) release the broach handle from the broach during surgery, leaving the broach in place while the handle is removed, so that a femoral prosthesis head may be attached to the broach for determining the proper size and orientation of the hip prosthesis; and (ii) release the broach quickly and multiple times from the broach handle in order to change broach sizes.

### SUMMARY OF THE INVENTION

The present invention provides a novel apparatus for use in preparing bone to receive a prosthesis. More particularly, the present invention provides a surgical broach handle for preparing the intramedullary canal of a femur to receive a femoral prosthesis with minimal soft tissue damage to the surgical site.

In one form of the present invention, there is provided a broach handle for releasable attachment to a broach, wherein the broach comprises a distal working end and a proximal connector end, wherein the proximal connector end comprises a bore extending distally into the broach and a finger extending proximally away from the broach, wherein the finger comprises a recess, the broach handle comprising:

a body having a distal end and a proximal end, wherein the distal end is longitudinally offset, and laterally offset, from the proximal end, and further wherein the distal end of the body comprises a finger for seating in the bore of the broach and bore for receiving the finger of the broach;

a linkage having a distal end and a proximal end and being pivotally connected to the body, wherein the distal end comprises a finger for selective disposition in the recess of the finger of the broach; and

a lever having a distal end and a proximal end and being pivotally connected to the body, wherein the distal end of the lever is arranged to manipulate the proximal end of the linkage;

whereby manipulation of the proximal end of the lever can lock and unlock a broach to the handle.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1-3 are schematic views showing a broach handle formed in accordance with the present invention, with a broach attached;

FIGS. 4-6 are schematic views showing a typical broach for use with the broach handle of the present invention;

FIGS. 7-10 are schematic views showing the body of the broach handle of the present invention;

FIGS. 11-13 are schematic views showing the linkage mechanism of the broach handle;

FIG. 14 is a schematic view showing the lever of the broach handle;

FIGS. 15 and 16 are schematic views showing the broach handle with a torque-bar attached; and

FIGS. 17 and 18 are schematic views showing the torque-bar shown in FIGS. 15 and 16.

### DETAILED DESCRIPTION OF THE PREFERRED EMBODIMENTS

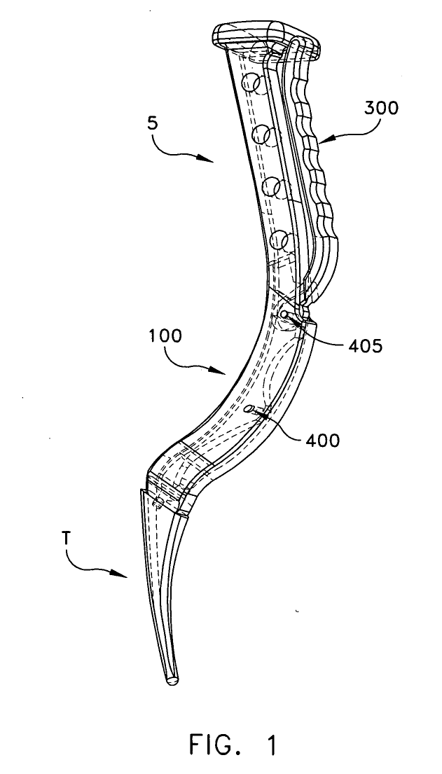

Looking now at FIGS. 1-3, in one preferred form of the present invention, there is provided a novel broach handle 5 for use in manipulating a conventional broach T. Broach handle 5 generally comprises a body 100, a linkage 200 and a lever 300.

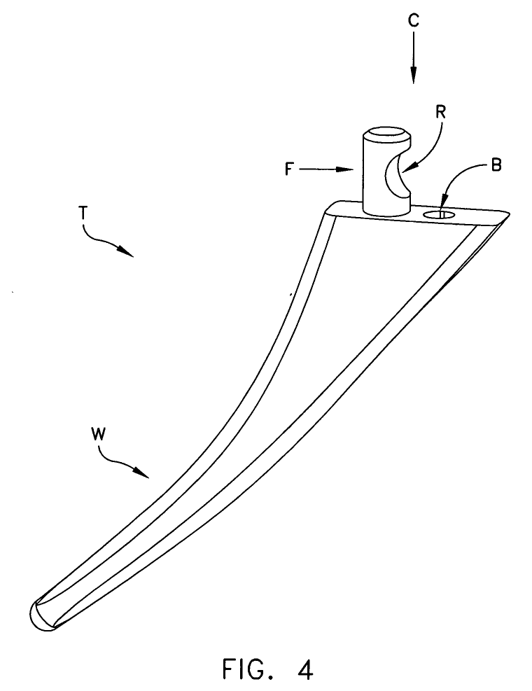

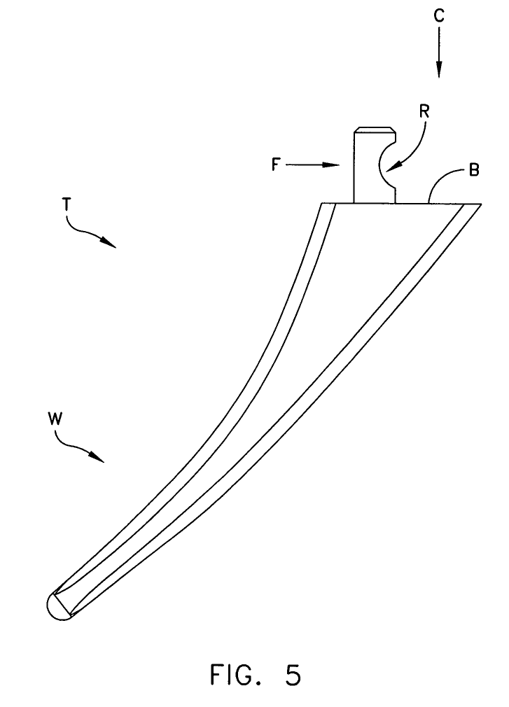

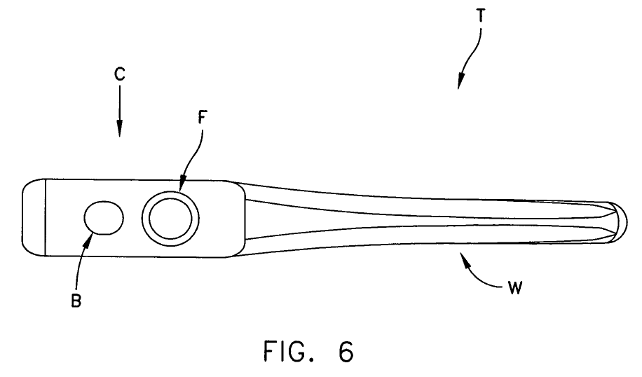

Broach T may be any broach compatible with the present invention. Looking now at FIGS. 1-3 and 4-6, broach T is generally characterized by a working end W and a connector C. Connector C is generally characterized by a bore B and a finger F including a recess R.

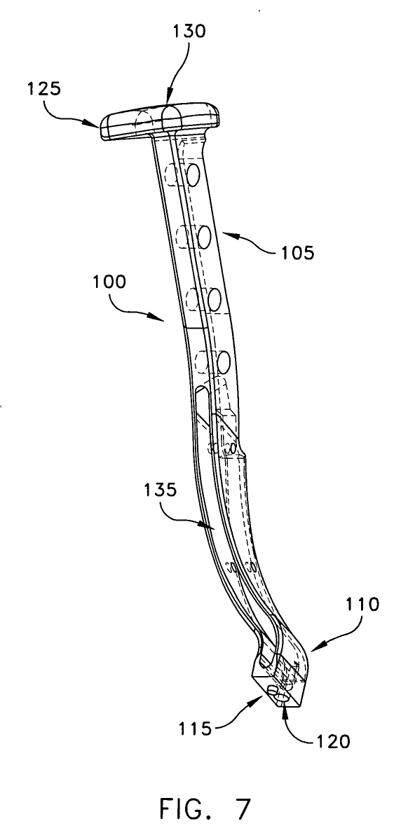

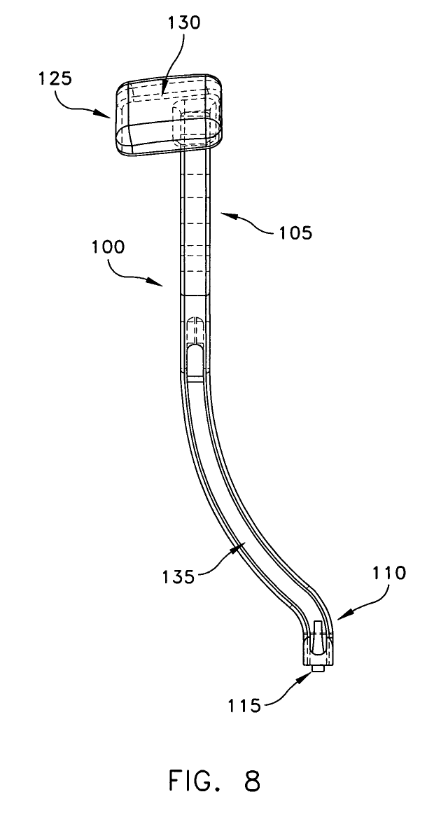

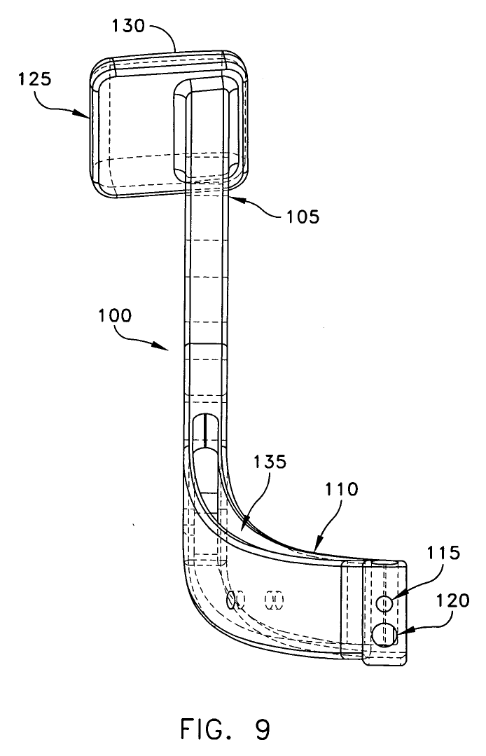

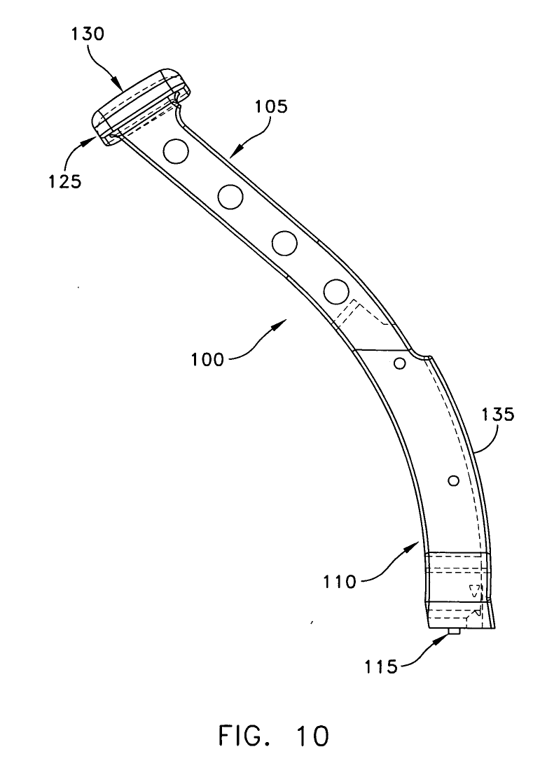

Body 100 is shown in FIGS. 1-3 and 7-10. Body 100 has a proximal end 105 and a distal end 110. As seen in FIG. 8, distal end 110 is laterally offset from proximal end 105. In addition, as seen in FIG. 10, distal end 110 is longitudinally offset from proximal end 105. Distal end 110 includes a finger 115 for disposition in bore B of broach T, and a recess 120 for receiving finger F of broach T. Proximal end 105 includes a strike plate 125 terminating in a strike surface 130. Strike plate 125 is preferably integral to handle 5 and canted in such a manner that strike surface 130 is in a plane which is set at substantially a right angle to the line of action towards broach T, when attached. This line of action is the direction of the vector forces directed from the strike plate to the attachment point of the broach. A slot 135 is formed in body 100.

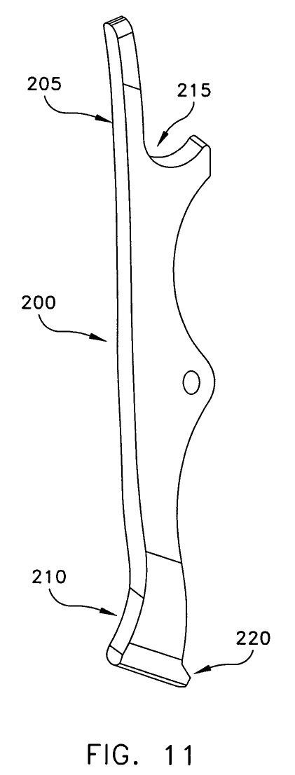

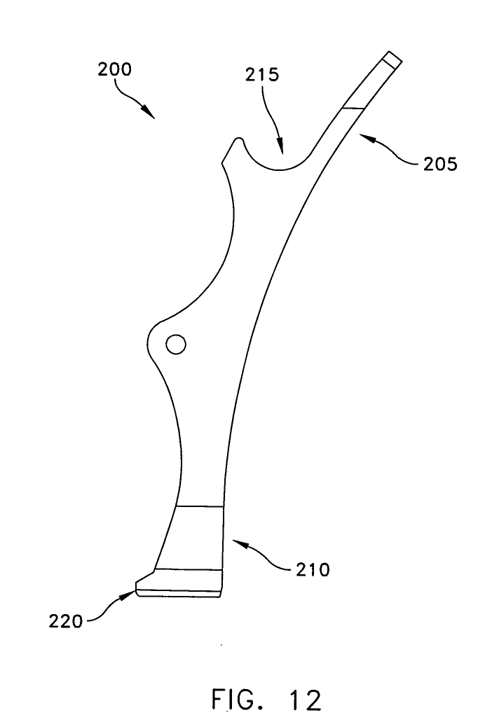

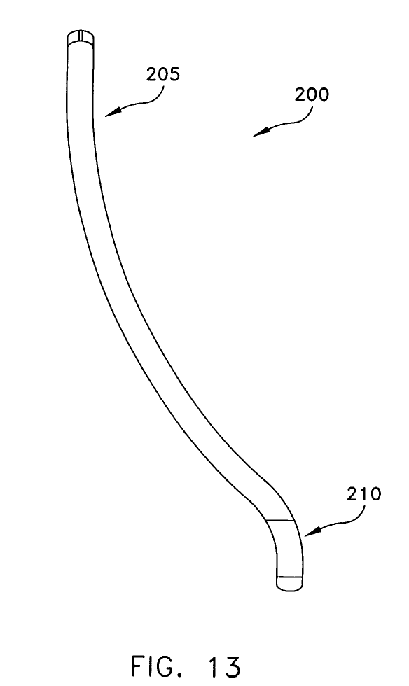

Linkage 200 is shown in FIGS. 1-3 and 11-13. Linkage 200 is generally characterized by a proximal end 205 and a distal end 210. Distal end 210 is laterally offset from proximal end 205 in a manner which corresponds to the extent of the lateral offset of the body's distal end 110 from the body's proximal end 105. Proximal end 205 includes a recess 215 for engagement by lever 300 as will hereinafter be discussed. Distal end 210 includes a finger 220 for selective disposition in recess R of finger F of broach T. Linkage 200 is sized to fit within slot 135 of body 100 as will hereinafter be discussed.

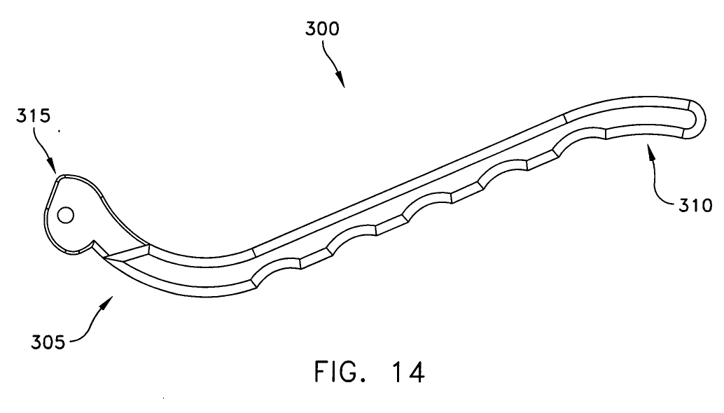

Lever 300 is shown in FIGS. 1-3 and 14. Lever 300 is generally characterized by a distal end 305 and a proximal end 310. Distal end 305 includes a cam mechanism 315 for engaging proximal end 205 of linkage 200.

As seen in the drawings, linkage 200 is disposed in body slot 135 and pivotally mounted to body 100 by a pin 400. Lever 300 has its distal end 305 disposed in body slot 135 and is pivotally mounted to body 100 by a pin 405.

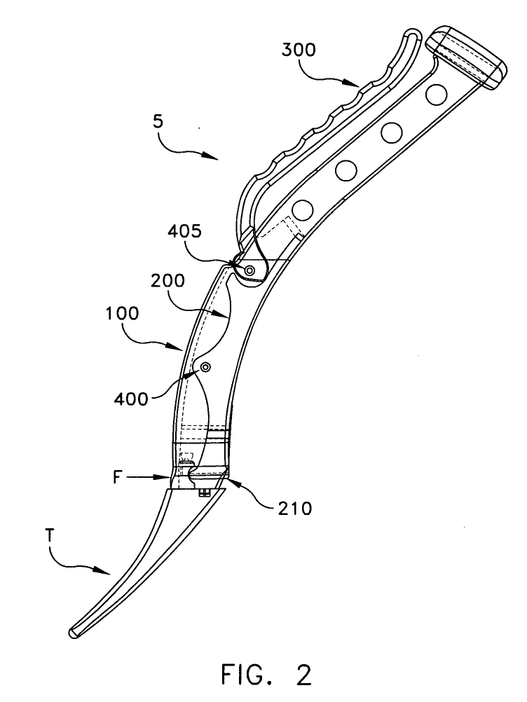

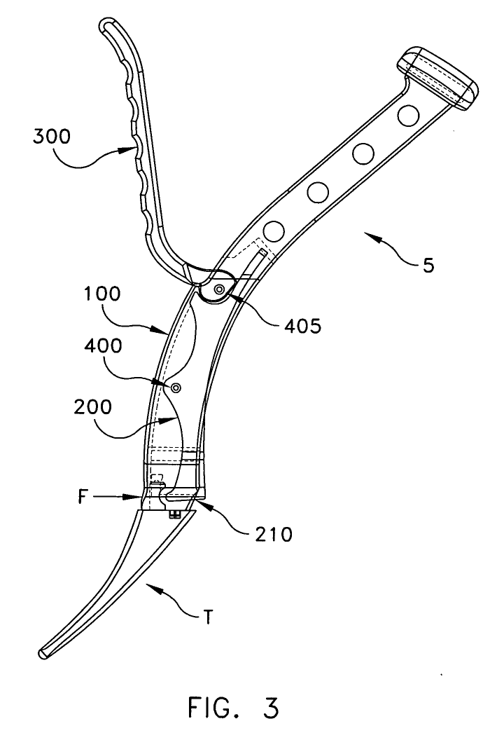

On account of the foregoing construction, when lever 300 is rotated counterclockwise (from the frame of reference of FIGS. 2 and 3), linkage 200 will be rotated counterclockwise, so that its distal end 210 is also rotated counterclockwise, whereby to permit a broach to be mounted or dismounted from the proximal end of body 100. More particularly, when lever 300 and linkage 200 are in the position shown in FIG. 3, broach T may be fit to the distal end of body 100 so that the broach's finger F is received in body recess 120 and body finger 115 is received in broach bore B.

Correspondingly, when lever 300 is moved clockwise (from the frame of reference of FIGS. 2 and 3), cam mechanism 315 on the distal end of lever 300 will cause linkage 200 to rotate clockwise, whereby to position linkage finger 220 in recess R of broach finger F, thereby locking broach T to the distal end of body 100.

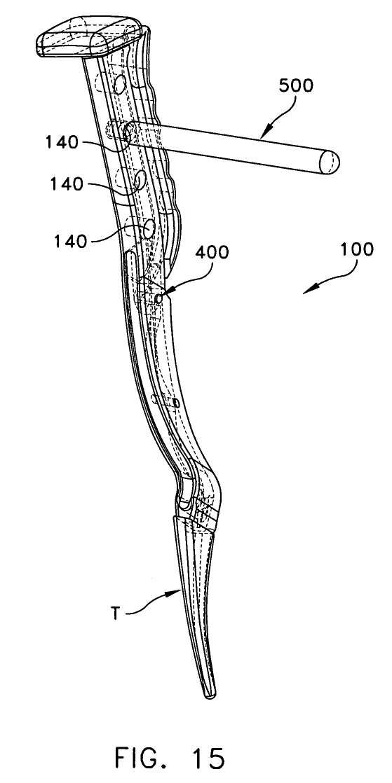

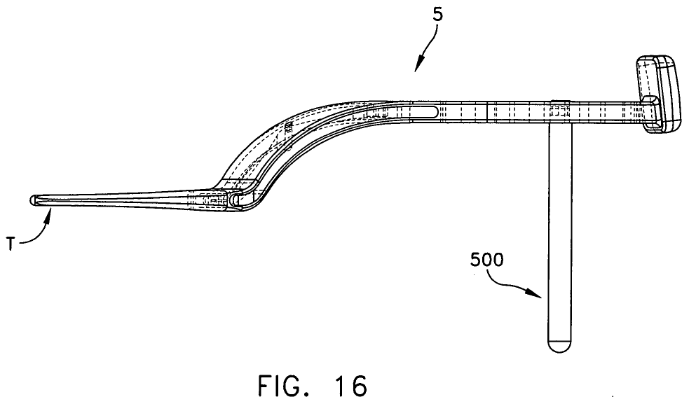

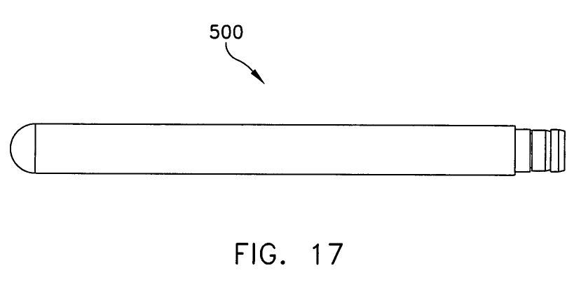

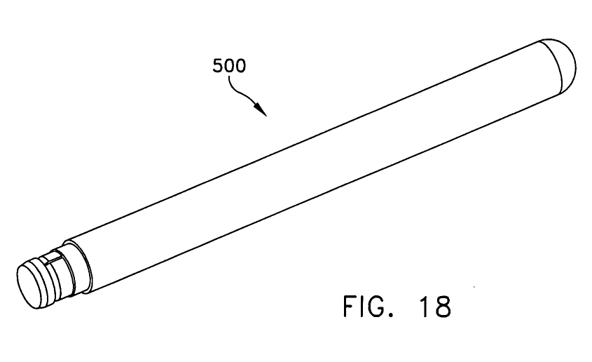

FIGS. 15-18 show a torque-bar 500 which may be inserted into a bore 140 formed in body 100. Torque-bar 500 may be used to manipulate handle 5 in ways well known in the art, e.g., to move handle 5 in a proximal direction or to apply torsion to the handle. Preferably a plurality of bores 140 are formed in body 100 so as to provide a range of possible positions for torque-bar 500. Bores 140 also help reduce the weight of body 100.

In one preferred form of the present invention, the novel broach handle is constructed of stainless steel. It will be appreciated that the present invention might alternatively be constructed out of other materials, e.g., plastic, other metals, etc.

Additional details regarding the construction of the broach handle shown in FIGS. 1-18 will now be provided.

Slot. The slot is located on one side of the narrow aspect of the handle's rectangular cross-section. The slot permits the attachment of the lever and the linkage at the proximal margin of the slot. The slot housing the distal end of the lever and the entire linkage components reside inside the slot at the distal end of the handle. A unique feature of the handle is its two compound curves which match the patient's anatomy, especially in a new search surgical technique called the anterior approach. There are holes through the handle proximally for attachment of the torque-bar.

Proximal pin holes. This pin is held in place by a through-hole across the entire handle and normal to the slots minor axis. The proximal pin holds the distal aspect of the lever in place and it acts as a fulcrum for its actuation.

Distal pin holes. This through-pair of holes in the handle holds the distal pin in place near the midpoint of the linkage in place and acts as a fulcrum for its actuation.

Lever. This behaves as a sea-saw or lever with a central fulcrum support.

Proximal finger grips. There are a total of five detents on the outside margin of the lever to improve the surgeon's grip and comfort.

Proximal flare. At the proximal-most aspect of the lever, the metal is flared out away from the handle. This prevents the surgeon's hand from slipping off the lever during broaching.

Fulcrum. This is located in the slot and held in place using the proximal pin described elsewhere in this document under the description of the handle. This fulcrum is located near the distal end of the lever so that large linear motion of the proximal lever results in small changes in the position of the distal lever. When the proximal end of the lever moves to the midline it is in the closed position. This moves the distal end of the lever laterally away from the midline of the slot.

Distal Cam. Located on the very distal end of the lever is cam geometry centered about the proximal pin in the slot of the handle. As the lever is closed, it rotates about the proximal fulcrum pin located in the slot. The radius of the lever cam contacts the linkage arm described elsewhere. As the lever is closed, the cam rotates and increases the length of the cam radius as measured from the central axis of the distal pin. This increasing cam radius then moves the proximal end of the linkage away from the midline of the slot, which in turn moves the distal end of the linkage into a closed position about the post on the broach. When the proximal end of the lever moves to the midline, it is in the closed position. This moves the distal end of the lever laterally away from the midline of the slot. When the proximal end of the lever moves to the midline, it is in the closed position. This moves the distal end of the lever laterally away from the midline of the slot.

Linkage. Like the lever, this is also a see-saw mechanism. The linkage rests in the slot of the handle.

Distal slot pin. The linkage is held in the slot using on through pin which passes through the handle and its slot near the distal end of the handle slot. When the lever is closed, the distal cam on the lever has its smallest effective radius in contact with the linkage. When the lever is open, the distal cam on the lever has its largest effective radius in contact with the linkage.

Open position. When the lever is in the open position, the distal bar feature on the linkage is moved away the midline of the handle and away from the slot on the proximal cutting broach. The cutting broach moves freely in this situation because the bar on the linkage is removed from the slot on the top of the cutting broach.

Closed position. When the lever is in the closed position, the distal bar feature on the linkage is moved towards the midline of the handle and towards the slot on the proximal cutting broach. The cutting broach is locked onto the broach handle in this situation because the bar on the linkage is pushed into the slot on the top of the cutting broach.

Cutting Broach. The particular broach in question here has an angled and flat proximal surface for mating to the broach handle. Normal to this proximal flat surface is a protruding pin. This pin has a semicircular notch located hear its proximal end. The linkage described above has a male semicircle of material to mate with the detent on the cutting broach. Other broach configurations exist in the marketplace, however, the linkage and lever construction of the present invention may also be used with these designs.

Torque-bar. This attaches normal to the long axis of the broach handle to help the surgeon resist torque loads seen during broaching.

### USE

In one preferred manner of use, lever 300 is set to its unlocked position (FIG. 3), a broach T is fit to the distal end of body 100, and lever 300 is set to its locked position (FIG. 2) so as to secure broach T to the distal end of handle 5. Handle 5 may then be used in ways well known in the art to prepare the femoral cavity using broach T. Significantly, the geometry of handle 5 facilitates preparation of the femoral cavity without impinging upon tissue even when used in a minimally invasive total hip procedure. If it should be determined that it is necessary to use a different broach T, the current broach T is dismounted from handle 5 by moving lever 300 to its unlocked position (FIG. 3), dismounting the current broach T, mounting a substitute broach T to the distal end of handle 5 and then moving lever 300 to its locked position (FIG. 2) so as to secure the new broach T in position.

If desired, handle 5 may be detached from broach T while the broach is in the femoral cavity. Significantly, the geometry of handle 5 facilitates removal of the handle from the broach, while the broach is position inside the bone, without impinging upon tissue even when used in a minimally invasive total hip procedure. Correspondingly, when the broach is thereafter to be reacquired by the handle, the geometry of handle 5 facilitates reattachment without impinging upon tissue even when used in a minimally invasive total hip procedure.

### MODIFICATIONS

While the present invention has been described in terms of certain exemplary preferred embodiments, it will be readily understood and appreciated by those skilled in the art that it is not so limited, and that many additions, deletions and modifications may be made to the preferred embodiments discussed herein without departing from the scope of the invention.

What is claimed is:

1. A broach handle for releasable attachment to a broach, wherein the broach comprises a distal working end and a proximal connector end, wherein the proximal connector end comprises a bore extending distally into the broach and a finger extending proximally away from the broach, wherein the finger comprises a recess, the broach handle comprising:
a body having a distal end and a proximal end, wherein the distal end is longitudinally offset, and laterally offset, from the proximal end, and further wherein the distal end of the body comprises a finger for seating in the bore of the broach and bore for receiving the finger of the broach;
a linkage having a distal end and a proximal end and being pivotally connected to the body, wherein the distal end comprises a finger for selective disposition in the recess of the finger of the broach; and
a lever having a distal end and a proximal end and being pivotally connected to the body, wherein the distal end of the lever is arranged to manipulate the proximal end of the linkage;
whereby manipulation of the proximal end of the lever can lock and unlock a broach to the handle.
